(19) United States
(12) Patent Application Publication
(10) Pub. No.: US 2025/0248636 A1
(43) Pub. Date: Aug. 7, 2025

(54) CORTICAL MAPPING FOR OPTIMAL BRAIN-COMPUTER INTERFACE PERFORMANCE

(71) Applicant: PRCEISION NEUROSCIENCE CORPORATION, New Yor, NY (US)

(72) Inventors: Benjamin I. RAPOPORT, New York, NY (US); Adam Poole, New York, NY (US); Morgan Lamarca, New York, NY (US); Craig Mermel, New York, NY (US)

(73) Assignee: PRCEISION NEUROSCIENCE CORPORATION, New York, NY (US)

(21) Appl. No.: 19/046,003

(22) Filed: Feb. 5, 2025

Related U.S. Application Data
(60) Provisional application No. 63/549,722, filed on Feb. 5, 2024.

(51) Int. Cl. A61B 5/374; A61B 5/293; A61B 5/377

(52) U.S. Cl. CPC A61B 5/374; A61B 5/293; A61B 5/377

(57) ABSTRACT

Systems and methods for cortical mapping to optimize placement of a neural interface in a brain of a patient are provided. A method for cortical mapping of a brain of a patient includes implanting an electrode array proximate to the brain of the patient at a position. The electrode array may include a flexible substrate and a plurality of electrodes arranged on the flexible substrate. The method may further include monitoring and electrophysiological mapping of the brain by causing the patient to perform, attempt to perform, or imagine performing an action over a period of time, recording, via the electrode array, neural activity exhibited by the patient in response to the action or imagined action performed by the patient, decoding the neural activity to determine a correspondence between the neural activity and the action, and determining a level of confidence, wherein the level of confidence is based on the correspondence.

### CROSS-REFERENCE TO RELATED APPLICATIONS

The present application claims priority to U.S. Provisional Patent Application No. 63/549,722, titled CORTICAL MAPPING FOR OPTIMAL BRAIN-COMPUTER INTERFACE PERFORMANCE, filed Feb. 5, 2024, which is hereby incorporated by reference herein in its entirety.

### TECHNICAL FIELD

Embodiments disclosed herein generally relate to systems and methods for cortical mapping to optimize placement of a neural interface in a brain of a patient.

### BACKGROUND

Brain-computer interfaces have shown promise as systems for restoring, replacing, and augmenting lost or impaired neurological function in a variety of contexts, including paralysis from stroke and spinal cord injury, blindness, and some forms of cognitive impairment. Multiple innovations over the past several decades have contributed to the potential of these neural interfaces, including advances in the areas of applied neuroscience and multichannel electrophysiology, mathematical and computational approaches to neural decoding, power-efficient custom electronics and the development of application-specific integrated circuits, as well as materials science and device packaging. Nevertheless, the practical impact of such systems remains limited, with only a small number of patients worldwide having received highly customized interfaces through clinical trials.

High bandwidth brain-computer interfaces are being developed to enable the bidirectional communication between the nervous system and external computer systems in order to assist, augment, or replace neurological function lost to disease or injury. A necessary capability of any brain-computer interface is the ability to accurately decode electrophysiologic signals recorded from individual neurons, or populations of neurons, and correlate such activity with one or more sensory stimuli or intended motor response. For example, such a system may record activity from the primary motor cortex in an animal or a paralyzed human patient and attempt to predict the actual or intended movement in a specific body part; or the system may record activity from the visual cortex and attempt to predict both the location and nature of the stimuli present in the patient's visual field.

Furthermore, brain-penetrating microelectrode arrays have facilitated high-spatial-resolution recordings for brain-computer interfaces, but at the cost of invasiveness and tissue damage that scale with the number of implanted electrodes. In some applications, softer electrodes have been used in brain-penetrating microelectrode arrays; however, it is not yet clear whether such approaches offer a substantially different tradeoff as compared to conventional brain-penetrating electrodes. For this reason, non-penetrating cortical surface microelectrodes represent a potentially attractive alternative and form the basis of the system described here. In practice, electrocorticography (ECoG) has already facilitated capture of high-quality signals for effective use in brain-computer interfaces in several applications, including motor and speech neural prostheses. Higher-spatial-resolution micro-electrocorticography (μECoG) therefore represents a promising combination of minimal invasiveness and improved signal quality. Therefore, it would be highly beneficial for neural devices to make use of non-penetrating cortical interfaces.

However, due to the neural device implant in the brain possibly being permanent or irreversible, diagnostic testing is desirable of temporary or reversible implants. The testing may assess responses to the temporary or reversible implants over time.

### SUMMARY

Accordingly, systems and methods for cortical mapping to optimize placement of a neural interface in a brain of a patient is provided. Mapping of temporary or reversible implants assists in placement of permanent implants such that the location of the neural interface does not need to be adjusted.

Described herein are high resolution, high channel count (HRHC) arrays manufactured using semiconductor microfabrication techniques that contain more than 1,000 electrodes, providing an unprecedented resolution for cortical surface mapping.

In some embodiments, a method for cortical mapping of a brain of a patient includes implanting an electrode array proximate to the brain of the patient at a first position, wherein the electrode array includes a flexible substrate and a plurality of electrodes arranged on the flexible substrate. The method includes monitoring electrophysiological activity of the brain by causing the patient to perform or imagine performing an action over a period of time, recording, via the electrode array, neural activity exhibited by the patient in response to the action performed or imagined by the patient, decoding the neural activity to determine a correspondence between the neural activity and the action, determining a level of confidence, wherein the level of confidence is based on the correspondence, and recording in a map generated via the cortical mapping, in association with the first position, both the level of confidence and the correspondence between the neural activity and the action.

In some embodiments, the method further includes moving the electrode array to a second position based on the level of confidence.

In some embodiments, the second position is adjacent to the first position.

In some embodiments, the method further includes removing the electrode array based on the level of confidence.

In some embodiments, the method further includes implanting a second electrode array proximate to the brain of the patient at a new position, wherein the second electrode array includes a flexible substrate and a plurality of electrodes arranged on the flexible substrate.

In some embodiments, the period of time is at least seven days.

In some embodiments, the action includes at least one of the patient speaking, the patient thinking about or imagining speaking, the patient moving a limb repeatedly over the period of time, the patient thinking about or imagining moving a limb repeatedly over the period of time, or the patient emotionally reacting to certain cues in a way that evoke an observable change in facial expression.

In some embodiments, the electrode array is configured for at least one of recording or stimulation.

In some embodiments, the method further includes implanting another electrode array at a third position.

In some embodiments, the decoding further includes training a machine learning model to decode neural activity of the action.

In some embodiments, the electrode array comprises a thin, flexible 1,024 channel array, wherein each channel corresponds to an electrode, and wherein each electrode has a diameter less than about 500 microns and is spaced from an adjacent electrode of the electrode array by less than about 1 mm.

In some embodiments, a method for cortical mapping of a brain of a patient includes implanting an electrode array proximate to the brain of the patient at a first position, wherein the electrode array includes a flexible substrate and a plurality of electrodes arranged on the flexible substrate. The method further includes stimulating the electrode array electrically, generating an electrical response from the electrode array based on the stimulating, generating a physiologic or behavioral response based on the electrical response, recording, via the electrode array, neural activity exhibited by the patient in response to the physiologic or behavioral response, decoding the neural activity to determine a correspondence between the neural activity and the physiologic or behavioral response, determining a level of confidence, wherein the level of confidence is based on the correspondence, and recording in a map generated via the cortical mapping, in association with the first position, both the level of confidence and the correspondence between the neural activity and the action.

In some embodiments, the method further includes moving the electrode array to a second position based on the level of confidence.

In some embodiments, the second position is adjacent to the first position.

In some embodiments, the method includes removing the electrode array based on the level of confidence.

In some embodiments, the method includes implanting a second electrode array proximate to the brain of the patient at a new position, wherein the second electrode array includes a flexible substrate and a plurality of electrodes arranged on the flexible substrate.

In some embodiments, the stimulating is performed repeatedly over a period of time.

In some embodiments, the period of time is at least seven days.

In some embodiments, the physiologic or behavioral response comprises the patient speaking, the patient imagining speaking, the patient moving a limb, or the patient imagining moving a limb repeatedly over a period of time.

In some embodiments, the electrode array is configured for at least one of recording or stimulation.

In some embodiments, the method further includes implanting another electrode array at a third position.

In some embodiments, the decoding further includes training a machine learning model to decode neural activity of the physiologic or behavioral response.

In some embodiments, the electrical response is a detectable potential on the electrode array.

In some embodiments, the electrode array comprises a thin, flexible 1,024 channel array, wherein each channel corresponds to an electrode, and wherein each electrode has a diameter less than about 500 microns and is spaced from an adjacent electrode of the electrode array by less than about 1 mm.

In some embodiments, a method for cortical mapping of a brain of a patient includes implanting a pair of electrode arrays proximate to a brain of the patient at first and second positions, respectively, wherein each of the plurality of electrode arrays includes a flexible substrate and a plurality of electrodes arranged on the flexible substrate, wherein the pair of electrode arrays are configured to function together. The method further includes monitoring electrophysiological activity of the brain by causing the patient to perform or imagine performing an action over a period of time, recording, via the pair of electrode arrays, neural activity exhibited by the patient in response to the action performed or imagined by the patient, decoding the neural activity to determine a correspondence between the neural activity and the action, determining a level of confidence, wherein the level of confidence is based on the correspondence, and recording in a map generated via the cortical mapping, in association with the first position, both the level of confidence and the correspondence between the neural activity and the action.

The method can further include stimulating the pair of electrode arrays electrically, generating an electrical response from the pair of electrode arrays based on the stimulating, generating a physiologic or behavioral response based on the electrical response, recording, via the pair of electrode arrays, neural activity exhibited by the patient in response to the physiologic or behavioral response, decoding the neural activity to determine a correspondence between the neural activity and the physiologic or behavioral response, determining a level of confidence, wherein the level of confidence is based on the correspondence, and recording in a map generated via the cortical mapping, in association with the first position, both the level of confidence and the correspondence between the neural activity and the action.

Further features of the present disclosure, as well as the structure and operation of various embodiments, are described in detail below with reference to the accompanying drawings. It is noted that the present disclosure is not limited to the specific embodiments described herein. Such embodiments are presented herein for illustrative purposes only. Additional embodiments will be apparent to persons skilled in the relevant art(s) based on the teachings contained herein.

### BRIEF DESCRIPTION OF THE FIGURES

The accompanying drawings, which are incorporated herein and form part of the specification, illustrate the present disclosure and, together with the description, further serve to explain the principles of the present disclosure and to enable a person skilled in the relevant art(s) to make and use embodiments described herein.

FIG. 1 illustrates an illustrative system including a neural device, according to illustrative embodiments of the present disclosure.

FIG. 2 illustrates an implantation process, according to illustrative embodiments of the present disclosure.

FIG. 3 is a flow diagram illustrating a method for cortical mapping to optimize placement of a neural interface in a brain of a patient, according to illustrative embodiments of the present disclosure.

FIG. 4 is a block diagram illustrating a computing device, according to illustrative embodiments of the present disclosure.

FIG. 5 is an image of a neural device implanted in a subject, according to illustrative embodiments of the present disclosure.

FIG. 6 is an image of a neural device electrode array under microscopy, according to illustrative embodiments of the present disclosure.

The features of the present disclosure will become more apparent from the detailed description set forth below when taken in conjunction with the drawings, in which like reference characters identify corresponding elements throughout. In the drawings, like reference numbers generally indicate identical, functionally similar, and/or structurally similar elements. Additionally, generally, the left-most digit(s) of a reference number identifies the drawing in which the reference number first appears. Unless otherwise indicated, the drawings provided throughout the disclosure should not be interpreted as to-scale drawings.

### DETAILED DESCRIPTION

The present disclosure is generally directed to systems and methods for cortical mapping to optimize placement of a neural interface in a brain of a patient. Cortical mapping is the process by which electrical recordings are performed at the cortical surface and associations are made between the anatomic locations of the recordings and external, internal, or environmental cues. Stimulation is applied to the cortical surface of the brain to identify functional areas for sensory, motor, or language functions. The process can analyze the brain and study the relationship between the structure of the brain and the corresponding systematic function. For example, during brain surgery, areas identified via cortical mapping are not resected such that the function of the area can function postoperatively. Performing cortical mapping with temporary or reversible implants prior to placing more permanent neural interface implants can be advantageous because it can ensure that the neural interfaces are positioned properly to read the desired regions of the cortical surface and have proper signal fidelity, avoiding the need for any post-implantation adjustments.

Progress toward the development of brain-computer interfaces has signaled the potential to restore, replace, and augment lost or impaired neurological function of humans in a variety of disease states. Existing approaches to developing high-bandwidth brain-computer interfaces rely on invasive surgical procedures or brain-penetrating electrodes, which limit addressable applications of the technology and the number of eligible patients. Similar approaches for assessing responses in patients has been performed in spinal cord stimulators to assess therapeutic responses or in electrophysiological mapping of the brain for seizure foci prior to epilepsy surgery.

Considerable progress has been made in recent decades in the tools used to interrogate human brain structure and function, and the relationships between them, in health and disease. It has long been appreciated that cortical “mapping,” a process of clearly defining the spatial extent of functional brain regions, leads to improved outcomes in the neurosurgical approach to brain tumors and other lesions in the vicinity of “eloquent” brain regions. The field of neuroimaging has witnessed improvements in spatial resolution over the past several decades, with improvements in magnetic resonance imaging (MRI) tractography influencing surgical planning, and functional MRI (fMRI) now capable of imaging voxels smaller than 1 mm³ in clinical practice. It is understood that imaging-based mapping techniques, even at the modern limits of resolution, do not provide real-time information on neural activity during surgery. This is because while imaging-based mapping techniques are helpful in pre-operative planning, they do not provide real-time information on neural activity during surgery. Electrophysiologic techniques for cortical mapping, on the other hand, can provide dynamic and real-time information. Advances in neurotechnology and artificial intelligence have made it possible to record and process large amounts of cortical electrophysiologic information at even smaller scales, but these advances have not yet been translated from basic science into clinical practice.

In recent years, progress toward clinically viable brain-computer interfaces has resulted in the development of several systems capable of recording human electrocorticographic activity at extremely fine spatial and temporal scales. Not all such systems are usable for standard clinical applications such as diagnostic electrophysiology, cortical mapping, or intraoperative monitoring, however. Intracortical microelectrodes, which have been used extensively in the field of brain-computer interfaces, are not ideally suited for intraoperative cortical mapping because they cause some trauma to the regions being mapped, and also because the acute response to insertion of such electrodes sometimes precludes their use in the period immediately following insertion. Micro-electrocorticography, the use of small, closely spaced surface electrodes, is a high-spatial-resolution version of a widely used technique for intraoperative cortical mapping. This technology may offer some of the benefits of real-time, high-resolution electrophysiology that have been developed for use in brain-computer interfaces, in ways that translate to practical, intraoperative patient care.

It is established in literature that passive intraoperative functional mapping using electrocorticography in the broadband gamma band (70-110 Hz) can safely and rapidly localize eloquent cortex in only a few minutes. The conventional electrocorticography (ECoG, or macro-ECoG) electrode arrays used currently in intraoperative monitoring are often restricted in feature sizes, typically with an electrode diameter of several mm and an inter-contact distance on the order of 1 cm. Given the limitations in existing ECoG technology and recent progress in the field of brain-computer interfaces, there has been growing interest in novel fabrication techniques allowing for the creation and testing of a new generation of micro-ECG arrays. In practice, these arrays provide superior performance compared to standard arrays, with early studies showing they reliably reveal cortical somatosensory response patterns which would not have been resolvable with conventional ECoG; more accurately predict spikes and classify and discriminate phonemes; and differentiate between ictal and interictal patterns that would show up as nearly identical spikes at the resolution of clinical electrocorticography. In addition, with modern computational power, this method avoids burdensome visual inspection and minimizes communication errors between neurosurgeon and neurophysiologist; recent results leverage an automated algorithm to create sensorimotor maps. These high-resolution arrays define state-of-the-art, but access has been historically limited, even within the academic sphere, due to the intricacies in fabrication.

Because neural interfaces require surgical intervention in order to be placed within the brain and necessarily interact with sensitive brain tissue, it is desirable to perform testing to ensure that a neural interface is placed at the proper location along the cortical surface. In particular, the electrode arrays of the neural interface should be placed such that they are able to sense and/or stimulate the targeted functional areas of the brain that are associated with the cognitive or motor functions that are being restored or replaced via the neural interface. The functional areas may include motor function, communication, movement, or speech.

For example, if a neural interface is being used in an application to restore hand motor function of a patient, the electrode array of the neural interface must be located at the brain region related to hand motor function in order to properly sense and/or stimulate the targeted function. Notably though, although the general location of each functional area of the brain is well-understood, it is impossible to predict the precise location and morphology of each functional area due to anatomic variation from individual-to-individual. Accordingly, without direct testing, the exact location of each functional area cannot be precisely known for each individual patient. That is, at a mm or sub-mm scale, the brain may be different between patients.

With conventional neural implants, the surgical team makes a best guess as to the location of the targeted functional area of the brain and then implants the neural implant at that location. Because conventional neural implants utilize penetrating electrodes, there is no opportunity to move or otherwise adjust the positioning of the neural implant because doing so would cause damage to the brain tissue. Therefore, if the positioning of a conventional neural implant is subsequently determined to be incorrect or non-ideal, the positioning of the implant cannot be adjusted to improve its performance. This inability to correct improperly positioned neural implants leads to a high rate of poor performance or failure in such implants.

Precise positioning of neural implants can be highly desirable because if the neural implant does not lay precisely over the targeted function area of the brain, it may be unable to fully sense the signals associated with the brain region (or, conversely, effectively and completely stimulate the brain region) and may also sense unrelated signals from adjacent brain regions that interfere with the ability to identify and decode the desired signals. Provided herein are techniques to determine the precise location of certain functional areas of the brain so that the neural implant is positioned at the correct location. Notably, the techniques described herein are not able to be performed by conventional penetrating electrode arrays.

In order to optimize the exact placement of the electrode array, electrophysiological mapping at a high resolution is preferred. Because the electrode array described herein uses non-penetrating electrodes, it is minimally invasive and, therefore, well suited for this type of cortical mapping.

### Neural Interfaces

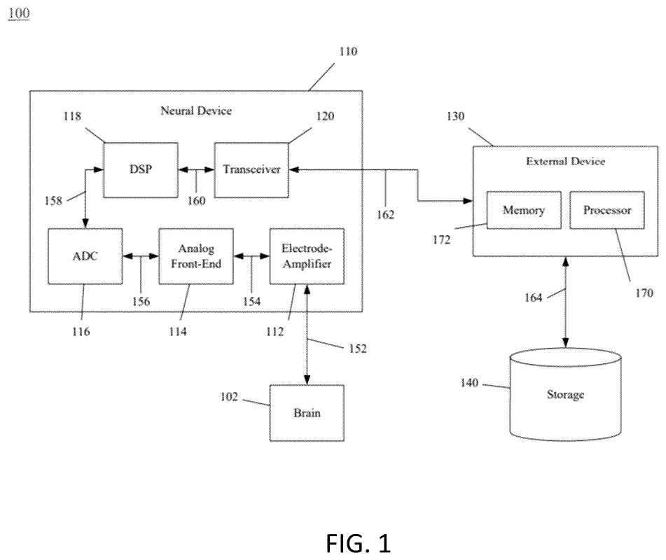

Referring now to FIG. 1, there is shown a diagram of an illustrative system 100 including a neural device 110 that is communicatively coupled to an external device 130. The external device 130 may include any device that the neural device 110 may be communicatively coupled, such as a computer system or mobile device (e.g., a tablet, a smartphone, a laptop, a desktop, a secure server, a smartwatch, a head-mounted virtual reality device, a head-mounted augmented reality device, or a smart inductive charger device). The external device 130 may include a processor 140 and a memory 142. In some embodiments, the computer system or mobile device may include a server or a cloud-based computing system. In some embodiments, the external device 130 may further include or be communicatively coupled to storage 140. In one embodiment, the storage 140 may include a database stored on the external device 130. In another embodiment, the storage 140 may include a cloud computing system (e.g., Amazon Web Services or Azure).

The neural device 110 may include a range of electrical or electronic components. In the illustrated embodiment, the neural device 110 includes an electrode-amplifier stage 112, an analog front-end stage 114, an analog-to-digital converter (ADC) stage 116, a digital signal processing (DSP) stage 118, and a transceiver stage 120 that are communicatively coupled together. The electrode-amplifier stage 112 may include an electrode array, such as is described below, that is able to physically interface with the brain 102 of the user in order to sense brain signals and/or apply electrical signals thereto.

The analog front-end stage 114 may be configured to amplify signals that are sensed from or applied to the brain 102, perform conditioning of the sensed or applied analog signals, perform analog filtering, and so on. The front-end stage 114 may include, for example, one or more application-specific integrated circuits (ASICs) or other electronics, such as the ADC stage 116. The ADC stage 116 may be configured to convert received analog signals to digital signals and/or convert received digital signals to an analog signal to be processed via the analog front-end stage 114 and then applied via the electrode-amplifier stage 112.

The DSP stage 118 may be configured to perform various DSP techniques, including multiplexing of digital signals and/or reducing the data rate by performing a feature extraction or data compression of the digitized signals received via the electrode-amplifier stage 112 and/or from the external device 130. For example, the DSP stage 118 may be configured to convert instructions from the external device 130 to a corresponding digital signal. The transceiver stage 120 may be configured to transfer data from the neural device 110 to the external device 130 located outside of the body of the user.

In various embodiments, the stages of the neural device 110 may provide unidirectional or bidirectional communications in half-or full-duplex mode (as indicated in FIG. 1) by and between the neural device 110 and the external device 130. In various embodiments, one or more of the stages may operate in a serial or parallel manner with other stages of the system 100. It may further be noted that the depicted architecture for the system 100 is simply intended for illustrative purposes and that the system 100 may be arranged differently (i.e., components or stages may be connected in different manners) or include additional components or stages.

The electrode-amplifier stage 112 may include an electrode array. The electrode array may include 1,024 channels (i.e., electrodes), but more or less may also be used. In some embodiments, the electrode array may be disposed on a substrate that is sufficiently thin and flexible to allow the electrode array to be inserted using minimally invasive techniques, including those described in Ho et al., which is incorporated by reference below. In some embodiments, each electrode of the electrode array has a diameter less than about 500 μm. In some embodiments, each electrode spaced from an adjacent electrode of the electrode array by less than about 1 mm. Additionally, the electrode array does not damage the brain of the patient and may be removed or repositioned if necessary. By removing or repositioning the electrode array, the performance of the neural interface may be improved. That is, the electrode array is more likely to be in a location that is more suitable to recording or stimulating that area of the brain.

Additional information regarding brain-computer interfaces described herein can be found in Ho et al., The Layer 7 Cortical Interface: A Scalable and Minimally Invasive Brain-Computer Interface Platform, bioRxiv 2022.01.02.474656; doi: https://doi.org/10.1101/2022.01.02.474656, which is hereby incorporated by reference herein in its entirety.

### Cortical Mapping

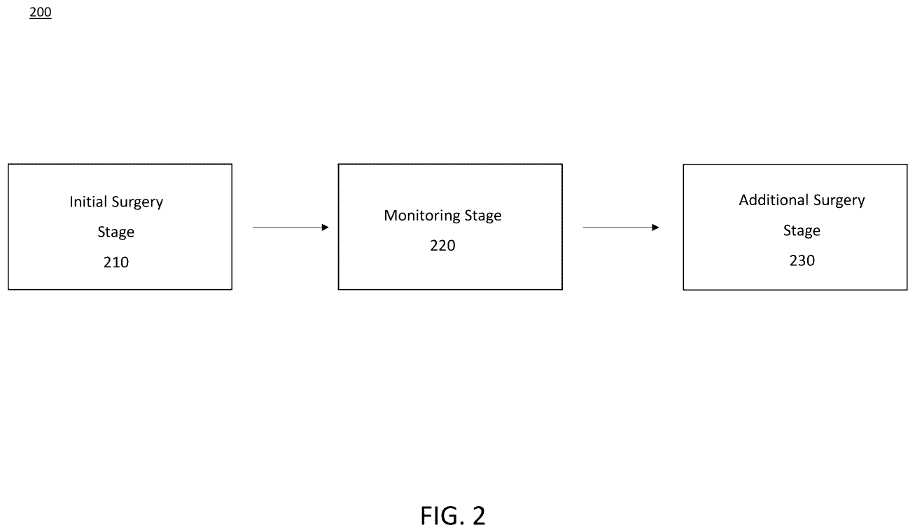

FIG. 2 illustrates an implantation process 200, according to some embodiments. The implantation process 200 may include at least three stages: an initial surgery stage 210, a monitoring in brain stage 220, and an additional surgery stage 230. As described above, the implantation process 200 can make use of neural devices 110 that include non-penetrating electrodes.

In the initial surgery stage 210, a neural device 110 is surgically placed at a location along the cortical surface of a patient. As described above, the initially placed neural device 110 can be a mapping neural device or an operational neural device. Initially, the electrode array of the neural device 110 is placed at the estimated location of the targeted functional area. The estimated position may depend on whether the patient is looking to restore specific functions, such as motor, communication, or the like. In some embodiments, temporary or “mapping” neural devices can be used for the initial surgery stage 210 and/or monitoring stage 220 prior to implantation of a semi-permanent or operational neural device. In some embodiments, the mapping neural devices may be substantially the same as the operational neural devices. In other embodiments, the mapping neural devices may be structurally and/or functionally different than the operational neural devices. In one embodiment, a neural device 110 used for the initial mapping procedure can include an electrode array that spans a larger area than the electrode array of the operational neural device. It can be beneficial for the mapping neural device to cover a large area than the final neural device to yield a larger area of the brain being mapped to assist in identifying the targeted functional area of the cortical surface. By mapping a larger area of the brain, one can more easily identify which location yields the best results depending the on the desired function from the patient. In this embodiment, the mapping electrode area can include larger electrodes (e.g., electrodes having larger diameters) than the electrode array of the operational neural device, electrodes that are spaced at larger distance than in the electrode array of the operational neural device, or a combination thereof. In one embodiment, the mapping neural device can include a 4×4 electrode array.

During the initial surgery stage 210, an initial check of the position of the electrodes may be performed. The surgeon may ask the patient to perform some actions during the placement procedure. Software may analyze the responses from the actions.

In the monitoring in brain stage 220, the patient and the signals sensed by the implanted neural device 110 are monitored for a period of time. In some embodiments, the patient can be monitored within the operating room and/or the patient may be moved to a monitoring unit. The period of time may vary depending on the patient, but can be a minimum of seven days, up to 30 days. During the monitoring stage 220, the patient may be asked to perform or imagine performing certain actions that relate to the area of the brain that the electrodes are inserted in. That is, if the electrodes are inserted in the speech area of the brain, the patient may be asked to do or imagine doing certain speech actions, such as talking, singing, speaking, or the like. Similarly, if the electrodes are inserted in the motor function area of the brain, the patient may be asked to do or imagine doing certain motor actions, such as moving a foot, hand, finger, leg, or the like.

As the actions are being performed by the patient, the neural device can detect the corresponding neural activity occurring contemporaneously at the location of the brain at which the electrode array of the neural device 110 is positioned. Software may be used for decoding such neural activity. During the monitoring, the signal quality, fidelity in correlating the performed actions with the sensed signals, and presence of any interfering signals are assessed. Based on these factors, the team can determine whether the location of the electrode array requires adjustment. Since the electrode array utilizes non-penetrating electrodes that do not damage the patient's brain, the location of the neural device may be adjusted as desired without risking permanent damage to the patient brain tissue.

The software may monitor certain parameters with respect to the actions that the patient is performing in the monitoring unit. For example, the parameters may relate to how many words the patient is typing, how many words the patient is speaking, how many steps the patient took, how many finger movements the patient made, poking a finger or foot with a pin, or the like including thinking about or imagining any of the foregoing actions.

In various implementations, response in the brain region being monitored can be stimulated using either indirect or direct techniques. For example, the patient may be asked to perform or think about the function associated with the targeted region of the brain, which should generate an electrical response in the targeted brain region. To illustrate, if the neural device 110 is being used in connection with a motor function (e.g., hand or foot movement), the patient can be asked to move or think about moving the associated body part. As another example, the relevant body part can be directly electrically stimulated, which should generate an electrical response (which is referred to as an “invoked potential”) in the targeted brain region. Depending on the strength and fidelity of the sensed signal, one can determine whether the positioning of the neural device 110 should be adjusted. The monitoring of the electrophysiological electrode may be done continuously during the monitoring stage 220.

As, described above, software may assist in decoding the neural activity, either stimulated directly or indirectly, versus surface electrical activity in the brain, based off the position that the electrodes are placed. The software may have the ability to decode intent, speech, communication, or the like, which all may be assessed in the monitoring stage 220. The software may decode the movement or pattern of the action. In the monitoring stage 220, the voltage of the electrode may be streaming. The software may use an algorithm to determine a pattern of activity and distinguish between when the patient is performing actions or not performing actions. For example, when a patient performs a function, a corresponding neural signal is generated. The neural signal is detectable by the neural device. When the patient is not performing the function, no neural signal should be generated. The performing versus non-performing functions may be compared and distinguished between by the software.

The software, in order to determine if the parameters are met, may perform a behavioral paradigm. The behavioral paradigm may be a model situation that mirrors the same critical setting of the patient (such as the same relationships between environment, behavior, and consequences for the patient) as the situations they are supposed to model. A confidence level may be determined from the model situation, based on the behavioral paradigm.

Specifically, when a patient speaks, the software may use machine learning (ML) algorithms to identify the patterns of speech. The electrical activity from the electrodes may correspond to speaking and the algorithm learns over time what electrical activity corresponds to speaking, for example. Since the electrical activity corresponded to speaking, the mapping of the electrode is deemed reliable, and the location of the electrode is a good position.

If the mapping is determined to not be reliable, then the location of the electrodes should be moved, which is further described in the additional surgery stage 230.

In the additional surgery stage 230, the electrodes may be removed entirely, the position may be adjusted, or placement of an implantable system may be performed. Based on the mapping and parameters determined in the monitoring stage 220, a doctor may determine whether the patient requires a neural prosthetic or not. That is, if the parameters met a certain threshold based on the mapping, it is likely that the location of the electrodes is correct. If the threshold was not met, it is likely that the location where the electrodes were placed was not accurate and requires either removal or repositioning.

As noted above, a 4×4 electrode array may be using for the cortical mapping prior to implantation of the final neural device. Depending on the accuracy of the position of the 4×4 electrode array, some of the electrode arrays may be repositioned or removed. For example, if only half of the 4×4 electrode array are yielding a signal that is performing at a good location, then the half of the electrode array that are not performing may be either removed or repositioned.

If the electrodes were not in the right position to improve overall function of the brain or restore or repair motor or communication functions of the patient, then the electrodes may be removed. The patient may decide to not implant any further electrodes as there may be too large of a risk to implant a permanent neural interface when the previous location of the electrode array was not accurate.

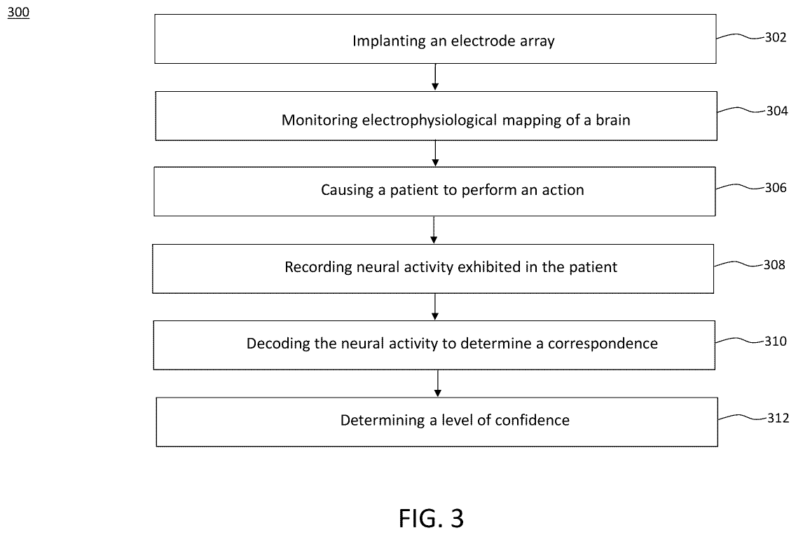

FIG. 3 is a flow diagram illustrating a method 300 for cortical mapping to optimize placement of a neural interface in the brain of a patient, according to some embodiments. In 302, an electrode array may be implanted proximate to the brain of the patient at a first position. The electrode array may include a flexible substrate and a plurality of electrodes arranged on the flexible substrate. For example, in the initial surgery stage 210, an electrode array may be implanted in the brain of a patient.

In 304, electrophysiological activity of the brain may be monitored. For example, in the monitoring stage 220, the electrophysiological activity of the brain of the patient may be monitored.

In 306, the patient may be caused to perform or imagine performing one or more actions over a period of time. For example, in the monitoring stage 220, the patient may be asked to perform or imagine performing actions during the patient's time in the monitoring stage 220. The actions may include speaking, moving a foot, moving an arm, or the like including imagining or thinking about performing any of the foregoing actions.

In 308, neural activity may be recorded via the electrode array. The neural activity may be exhibited by the patient in response to the action performed or imagined by the patient. For example, in the monitoring stage 220, neural activity of the patient may be recorded. The neural activity of the patient may be in response to the action performed or imagined in step 306 such as speaking, moving a foot, moving an arm, or the like.

In 310, the neural activity may be decoded to determine a correspondence between the neural activity and the action. For example, in the monitoring stage 220, software may decode the neural activity of the patient. The software may determine a correspondence between the neural activity relating to the action performed or imagined in step 306 such as speaking, moving a foot, moving an arm, or the like.

In 312, a level of confidence may be determined. The level of confidence may be based on the correspondence. For example, in the monitoring stage 220, software may determine a level of confidence. The level of confidence may be based on the correspondence between the neural activity relating to the action performed in step 306 such as speaking, moving a foot, moving an arm, or the like.

The method 300 can further include recording in a map generated via the cortical mapping, in association with the first position, both the level of confidence and the correspondence between the neural activity and the action performed or imagined in step 306. The level of confidence may be the level of confidence determined in step 312. The correspondence between the neural activity and the action may be the correspondence between the neural activity and the action determined in step 310.

### Neural Interface Computer Systems

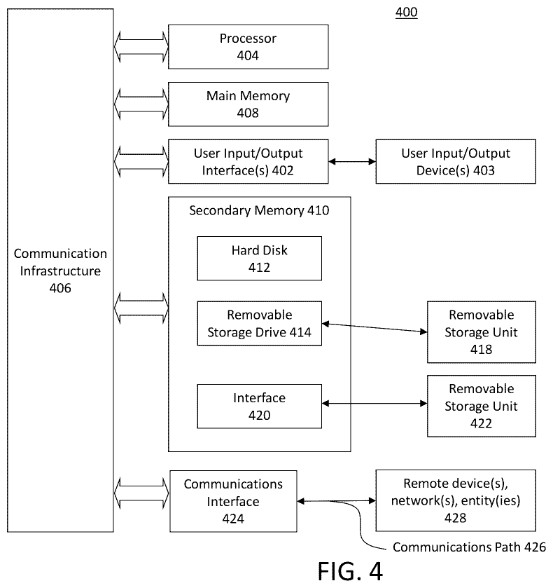

Various embodiments may be implemented, for example, using one or more well-known computer systems, such as computer system 400 shown in FIG. 4. For example, the system 100 may be implemented using combinations or sub-combinations of computer system 400. Also, or alternatively, one or more computer systems 400 may be used, for example, to implement any of the embodiments discussed herein, as well as combinations and sub-combinations thereof.

Computer system 400 may include one or more processors (also called central processing units, or CPUs), such as a processor 404. Processor 404 may be connected to a communication infrastructure or bus 406.

Computer system 400 may also include user input/output device(s) 403, such as monitors, keyboards, pointing devices, etc., which may communicate with communication infrastructure 406 through user input/output interface(s) 402.

One or more of processors 404 may be a graphics processing unit (GPU). In an embodiment, a GPU may be a processor that is a specialized electronic circuit designed to process mathematically intensive applications. The GPU may have a parallel structure that is efficient for parallel processing of large blocks of data, such as mathematically intensive data common to computer graphics applications, images, videos, etc.

Computer system 400 may also include a main or primary memory 408, such as random-access memory (RAM). Main memory 408 may include one or more levels of cache. Main memory 408 may have stored therein control logic (i.e., computer software) and/or data.

Computer system 400 may also include one or more secondary storage devices or memory 410. Secondary memory 410 may include, for example, a hard disk drive 412 and/or a removable storage device or drive 414. Removable storage drive 414 may be a floppy disk drive, a magnetic tape drive, a compact disk drive, an optical storage device, tape backup device, and/or any other storage device/drive.

Removable storage drive 414 may interact with a removable storage unit 418. Removable storage unit 418 may include a computer usable or readable storage device having stored thereon computer software (control logic) and/or data. Removable storage unit 418 may be a floppy disk, magnetic tape, compact disk, DVD, optical storage disk, and/any other computer data storage device. Removable storage drive 414 may read from and/or write to removable storage unit 418.

Secondary memory 410 may include other means, devices, components, instrumentalities or other approaches for allowing computer programs and/or other instructions and/or data to be accessed by computer system 400. Such means, devices, components, instrumentalities or other approaches may include, for example, a removable storage unit 422 and an interface 420. Examples of the removable storage unit 422 and the interface 420 may include a program cartridge and cartridge interface (such as that found in video game devices), a removable memory chip (such as an EPROM or PROM) and associated socket, a memory stick and USB or other port, a memory card and associated memory card slot, and/or any other removable storage unit and associated interface.

Computer system 400 may further include a communication or network interface 424. Communication interface 424 may enable computer system 400 to communicate and interact with any combination of external devices, external networks, external entities, etc. (individually and collectively referenced by reference number 428). For example, communication interface 424 may allow computer system 400 to communicate with external or remote devices 428 over communications path 426, which may be wired and/or wireless (or a combination thereof), and which may include any combination of LANs, WANs, the Internet, etc. Control logic and/or data may be transmitted to and from computer system 400 via communication path 426.

Computer system 400 may also be any of a personal digital assistant (PDA), desktop workstation, laptop or notebook computer, netbook, tablet, smart phone, smart watch or other wearable, appliance, part of the Internet of Things, and/or embedded system, to name a few non-limiting examples, or any combination thereof.

Computer system 400 may be a client or server, accessing or hosting any applications and/or data through any delivery paradigm, including but not limited to remote or distributed cloud computing solutions; local or on-premises software (“on-premise” cloud-based solutions); “as a service” models (e.g., content as a service (CaaS), digital content as a service (DCaaS), software as a service (SaaS), managed software as a service (MSaaS), platform as a service (PaaS), desktop as a service (DaaS), framework as a service (FaaS), backend as a service (BaaS), mobile backend as a service (MBaaS), infrastructure as a service (IaaS), etc.); and/or a hybrid model including any combination of the foregoing examples or other services or delivery paradigms.

Any applicable data structures, file formats, and schemas in computer system 400 may be derived from standards including but not limited to JavaScript Object Notation (JSON), Extensible Markup Language (XML), Yet Another Markup Language (YAML), Extensible Hypertext Markup Language (XHTML), Wireless Markup Language (WML), MessagePack, XML User Interface Language (XUL), or any other functionally similar representations alone or in combination. Alternatively, proprietary data structures, formats or schemas may be used, either exclusively or in combination with known or open standards.

In some embodiments, a tangible, non-transitory apparatus or article of manufacture comprising a tangible, non-transitory computer useable or readable medium having control logic (software) stored thereon may also be referred to herein as a computer program product or program storage device. This includes, but is not limited to, computer system 400, main memory 408, secondary memory 410, and removable storage units 418 and 422, as well as tangible articles of manufacture embodying any combination of the foregoing. Such control logic, when executed by one or more data processing devices (such as computer system 400 or processor(s) 404), may cause such data processing devices to operate as described herein.

Based on the teachings contained in this disclosure, it will be apparent to persons skilled in the relevant art(s) how to make and use embodiments of this disclosure using data processing devices, computer systems and/or computer architectures other than that shown in FIG. 4. In particular, embodiments may operate with software, hardware, and/or operating system implementations other than those described herein.

### Illustrative In-Human Cortical Mapping Study

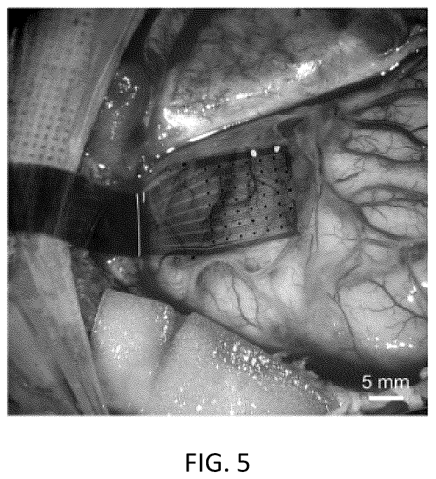

A study was conducted in the operating room during the portion of the procedure involving electrophysiologic cortical mapping. Study investigators routinely perform sensorimotor or language mapping on craniotomies involving resection of intrinsic brain tumors adjacent to or involving eloquent brain regions. Typically, a standard electrode grid array, such as a 4-, 6-, or 8-contact linear array, is placed over the area of interest that is being assessed, and cortical potentials are recorded over a period of seconds to minutes. In sensorimotor mapping, an electrical stimulus is applied to a nerve of the upper or lower extremity, such as the median nerve, and time-locked recordings are typically obtained from the cortical array. An illustrative embodiment of the HRHC neural interface device described herein and used in the study is shown in FIG. 5.

In language mapping, a transient electrical stimulus is applied to the region of interest (such as Broca's area, Wernicke's area, primary motor cortex, or adjacent regions) using a handheld probe. Typical stimulation parameters are; Pulse type: Monophasic anodal, Train pulse count: N=5, Pulse interval: 2.0 ms, Pulse duration: 0.2 ms, Stim rate: 4 Hz, Intensity: Surgeon-guided up to 15 mA. In collaboration with a neuropsychologist, the abilities of the patient to speak, read, comprehend and repeat are continuously assessed during stimulation and surgical manipulation. Changes associated with focal stimulation or manipulation are used to infer the functional role of the regions being stimulated or manipulated. Standard language mapping paradigms are used, and these have been described elsewhere.

The study team awaited the onset of this portion of the procedure, and once standard mapping was complete, the sterile HRHC array and connector cables were brought onto the surgical field. The recording procedure was repeated using the investigational device, connected to a system of digital amplifiers, and segments of each procedure were documented using videography over the surgical field. The location of the study electrode array on the brain was photographed both before and after array placement, and these images were retained for a variety of purposes, including digital data overlay during visualization, analysis, and post-processing.

During awake language mapping, auditory cues provided by an examiner or visual cues (single words) presented on a screen instructed the patient to speak individual words. The cues and the full auditory output of the patient were recorded and time-synchronized with electrophysiologic data for offline analysis.

A maximum of 15 minutes were used intraoperatively for the study procedure. Use of the device did not affect or change the surgical plan or intended procedure. All subjects were followed per standard of care by study personnel working alongside the clinical care teams. Any incidence of adverse events, such as infection or unexpected neurologic deficit, was noted.

The illustrative HRHC array used in this study was a thin-film electrode array containing 1024 contacts: 977 at 50 μm diameter, 42 at 380 μm, and 5 at 500 μm. The electrode pitch is 400 μm and the contact thickness is 480 nm. It consists of a polyimide substrate with platinum electrodes and associated electrically connecting traces, and an additional insulating layer of polyimide, totaling 22 microns in thickness.

The thin-film array is bonded to an interposer, which is in turn connected to recording boards that connect to a recording controller via cables extending from the package enclosure. In the present study, these inputs are processed via Intan recording software and visualized in real time via a custom software interface.

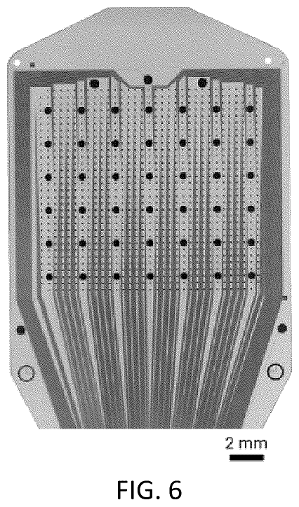

An image of the illustrative microelectrode array used in the study is shown in FIG. 6. The microelectrode arrays were fabricated on 6-8″ wafers using a spin-on polyimide. The fabrication process briefly comprised spin-coating, soft-bake, and vacuum cure of an approximately 10 μm layer of polyimide; photolithographic patterning, deposition, and liftoff of 20 nm/100 nm/200 nm/150 nm Ti/Pt/Au/Pt trace metal; O2 plasma treatment of the polyimide surface; spin-coating, soft-bake, and vacuum cure of an approximately 10 μm layer of polyimide; hard mask deposition and patterning for polyimide outline and electrode site opening; polyimide etch and electrode surface exposure in O2/SF6 plasma; hard mask strip; photolithographic patterning, deposition, and liftoff of 20 nm/20 nm/500 nm of Ti/Pt/Au bond pad metallization; and O2 plasma post-clean of the polyimide surface. Following microfabrication, devices were released in deionized water, optically inspected for trace, electrode, and pad defects, dehydration baked, and thermocompression bonded to an organic interposer using a flip-chip tool. The organic interposer printed circuit board (PCB) was fabricated using a typical high-density interconnect (HDI) PCB fabrication process, using an FR4 type epoxy laminate and typical HDI features such as blind and buried vias and via-in-pad structures. The PCB pads were coated with an industry standard immersion gold surface finish, to enable bonding to an oxidation-free surface for thermocompression bonding of the array directly to the PCB as described above. High density connectors were used to mate the interposer to custom Intan RHD2164-based digital interface boards with 128 channels per SPI output cable. A total of 8 digital interface boards per array were attached via SPI interface to the Intan 1024ch recording controller.

During central sulcus localization, electrical stimulation was applied to the median, ulnar, or tibial nerve via subdermal needle electrodes. Typical parameters were single pulse, 2-5 Hz frequency, 200 ms pulse width, and 5-20 mA amplitude, with precise values determined intraoperatively using standard-of-care methods with strip electrodes. For each stimulation pulse, a time-locked TTL signal was generated and co-recorded with the neural signals. The somatosensory evoked potentials (SSEPs) were then computed in real-time as the time-locked average of the neural signals over 128 trials.

For language mapping, patient vocalization was recorded using a microphone, and time synchronized to the neural recordings. Vocalization onsets were determined manually post-hoc via both audio playback and audio waveforms. To compute event-related beta band activity, the 20 kHz neural signals were first downsampled to 4 kHz, and then bandpass filtered for 13 to 30 Hz signals. The analytic amplitude was then computed by taking the absolute value of the Hilbert transform. The resultant beta band amplitude was further z-scored, using 0.75 seconds of event-free recordings per trial as baseline. Trials were excluded when a z-scored activity exceeded 10 on any sample. Spectrograms were computed for 4 kHz neural data using a Hann window, with 256 samples per segment and 128 samples of overlapping between segments. Event-related power spectral densities were computed for 4 kHz neural signals using Welch's method, for 0.5 seconds of baseline, 0.5 seconds of word display, and 0.5 seconds of word utterance.

Eight patients were consented and enrolled in this study from April 2023 to October 2024. Mean age at enrollment was 54±20 years and 75% of patients were male. Four (50%) of the cases involved asleep motor mapping with phase reversal, and four (50%) involved awake language mapping. In all cases the electrode array was deployed and removed without issue.

The standard electrode array was then replaced by the HRHC electrode array. The electrodes on the HRHC electrode array are 50 μm in diameter and 400 μm in pitch, in contrast to the standard strip array, where the electrodes were 4 mm in diameter and separated by 10 mm. Electrode impedances were measured after the electrode array was placed onto the target location, and electrodes within 1 to 4000 kΩ (measured at 1 kHz) were included for subsequent analyses. The somatosensory evoked potential (SSEP) recorded on the strip array was simulated as the mean of two electrode groups on the HRHC array. The reversal of phase across the central sulcus, from precentral gyrus to postcentral gyrus, was apparent across the majority of the electrodes in the HRHC array, identifying the region of each microelectrode as falling either anterior (motor) or posterior (sensory) to the central sulcus; a subset of the electrodes in the array outlined an isoelectric contour corresponding to the central sulcus itself. The resulting two-dimensional display of “phase reversal” constitutes a functional map of the underlying cortical surface, identifying one region as upper extremity primary somatosensory cortex (postcentral gyrus), one region as a corresponding region of primary motor cortex (precentral gyrus), and one region as a functional boundary between these two regions, overlying the anatomic central sulcus.

In standard central sulcus localization procedures, SSEPs measured on grid or strip electrodes are canonically visualized as a grid or column of averaged waveforms. As electrode count and density scales, the resolution and redundancy of information may be leveraged for intuitive, accurate, and rapid localization of the central sulcus. An example is visualization via voltage snapshots. After identifying the timing of clinically significant evoked responses, the corresponding 2-dimensional voltage heatmap can be created without further signal processing or computation, which generates the isoelectric contour corresponding to the central sulcus.

In several patients we recorded electrical activity while the patient underwent awake counting or word repetition tasks. When the HRHC array was placed on Broca's area, we observed consistent modulation of neural activity in the 1.5 seconds following word display, when the patient was not yet instructed to repeat the word, indicating speech planning. Between different words, the spatial distribution and temporal dynamics differ vastly, the patterns of which could only be elucidated through high resolution electrocorticography. Similar to previous studies, broadband power increase and alpha/beta band power decrease were observed immediately after speech utterance. In addition, using simple classifiers trained using only minutes of speech data, we achieved 80-90% accuracy in decoding speech intent in these patients even when the array was placed on the superior temporal gyrus, which is less typically used for speech decoding compared to the motor cortex, due to the more complex spatiotemporal neural characteristics.

Accordingly, the safety and feasibility of using a novel, high-spatial-resolution, thin-film cortical surface array was demonstrated in humans. The arrays described herein have been used in the context of intraoperative functional mapping of the cortical surface during surgery involving eloquent brain regions. The resulting electrophysiologic data illustrate the practicality and utility of high-spatial-resolution and high-channel count electrode arrays in intraoperative cortical mapping, to define and assess the integrity of functional regions such as motor-, somatosensory-, and language-related cortical areas in real time during neurosurgical operations with the potential to compromise those areas. The volume and nature of the data obtained from these arrays may enable us to refine our approach to intraoperative cortical mapping in a number of ways, by rendering two-dimensional electrophysiologic maps of the two-dimensional cortical surface; defining functional boundaries at microsurgical resolution; and facilitating systematic, unbiased, time-efficient language mapping.

Additional details regarding illustrative techniques for in-human cortical mapping can be found in Peter E Konrad et al., First-in-human experience performing high-resolution cortical mapping using a novel microelectrode array containing 1,024 electrodes, 2025 J. Neural Eng., DOI 10.1088/1741-2552/adaeed, which is hereby incorporated by reference herein in its entirety.

While the foregoing is directed to embodiments described herein, other and further embodiments may be devised without departing from the basic scope thereof. For example, aspects of the present disclosure may be implemented in hardware or software or a combination of hardware and software. One embodiment described herein may be implemented as a program product for use with a computer system. The program(s) of the program product define functions of the embodiments (including the methods described herein) and may be contained on a variety of computer-readable storage media. Illustrative computer-readable storage media include, but are not limited to: (i) non-writable storage media (e.g., read-only memory (ROM) devices within a computer, such as CD-ROM disks readably by a CD-ROM drive, flash memory, ROM chips, or any type of solid-state non-volatile memory) on which information is permanently stored; and (ii) writable storage media (e.g., floppy disks within a diskette drive or hard-disk drive or any type of solid state random-access memory) on which alterable information is stored. Such computer-readable storage media, when carrying computer-readable instructions that direct the functions of the disclosed embodiments, are embodiments of the present disclosure.

It will be appreciated to those skilled in the art that the preceding examples are exemplary and not limiting. It is intended that all permutations, enhancements, equivalents, and improvements thereto are apparent to those skilled in the art upon a reading of the specification and a study of the drawings are included within the true spirit and scope of the present disclosure. It is therefore intended that the following appended claims include all such modifications, permutations, and equivalents as fall within the true spirit and scope of these teachings.

1. A method for cortical mapping of a brain of a patient comprising:
implanting an electrode array proximate to the brain of the patient at a first position, wherein the electrode array comprises a flexible substrate and a plurality of electrodes arranged on the flexible substrate; and
monitoring electrophysiological activity of the brain by:
causing the patient to perform or imagine performing an action over a period of time,
recording, via the electrode array, neural activity exhibited by the patient in response to the action performed or imagined by the patient,
decoding the neural activity to determine a correspondence between the neural activity and the action,
determining a level of confidence, wherein the level of confidence is based on the correspondence, and
recording in a map generated via the cortical mapping, in association with the first position, both the level of confidence and the correspondence between the neural activity and the action.

2. The method of claim 1, further comprising:
moving the electrode array to a second position based on the level of confidence.

3. The method of claim 2, wherein the second position is adjacent to the first position.

4. The method of claim 1, further comprising:
removing the electrode array based on the level of confidence.

5. The method of claim 4, further comprising:
implanting a second electrode array proximate to the brain of the patient at a new position, wherein the second electrode array comprises a flexible substrate and a plurality of electrodes arranged on the flexible substrate.

6. The method of claim 1, wherein the period of time is at least seven days.

7. The method of claim 1, wherein the action comprises at least one of the patient speaking, the patient thinking about or imagining speaking, the patient moving a limb repeatedly over the period of time, the patient thinking about or imagining moving a limb repeatedly over the period of time, or the patient emotionally reacting to certain cues in a way that evoke an observable change in facial expression.

8. The method of claim 1, wherein the electrode array is configured for at least one of recording or stimulation.

9. The method of claim 1, further comprising:
implanting another electrode array at a third position.

10. The method of claim 1, wherein the decoding further comprises:
training a machine learning model to decode neural activity of the action.

11. The method of claim 1, wherein the electrode array comprises:
a 1,024 channel array,
wherein the 1,024 channel array is disposed on a thin and flexible substrate,
wherein each channel corresponds to an electrode, and
wherein each electrode has a diameter less than about 500 microns and is spaced from an adjacent electrode of the electrode array by less than about 1 mm.

12. A method for cortical mapping of a brain of a patient comprising:
implanting an electrode array proximate to the brain of the patient at a first position, wherein the electrode array comprises a flexible substrate and a plurality of electrodes arranged on the flexible substrate;
stimulating the electrode array electrically;
generating an electrical response from the electrode array based on the stimulating;
generating a physiologic or behavioral response based on the electrical response;
recording, via the electrode array, neural activity exhibited by the patient in response to the physiologic or behavioral response;
decoding the neural activity to determine a correspondence between the neural activity and the physiologic or behavioral response,
determining a level of confidence, wherein the level of confidence is based on the correspondence, and
recording in a map generated via the cortical mapping, in association with the first position, both the level of confidence and the correspondence between the neural activity and the physiologic or behavioral response.

13. The method of claim 12, further comprising:
moving the electrode array to a second position based on the level of confidence.

14. The method of claim 13, wherein the second position is adjacent to the first position.

15. The method of claim 12, further comprising:
removing the electrode array based on the level of confidence.

16. The method of claim 15, further comprising:
implanting a second electrode array proximate to the brain of the patient at a new position, wherein the second electrode array comprises a flexible substrate and a plurality of electrodes arranged on the flexible substrate.

17. The method of claim 12, wherein the stimulating is performed repeatedly over a period of time.

18. The method of claim 17, wherein the period of time is at least seven days.

19. The method of claim 12, wherein the physiologic or behavioral response comprises the patient speaking, the patient imagining speaking, the patient moving a limb, or the patient imagining moving a limb repeatedly over a period of time.

20. The method of claim 12, wherein the electrode array is configured for at least one of recording or stimulation.

21. The method of claim 12, further comprising:
implanting another electrode array at a third position.

22. The method of claim 12, wherein the decoding further comprises:
training a machine learning model to decode neural activity of the physiologic or behavioral response.

23. The method of claim 12, wherein the electrical response is a detectable potential on the electrode array.

24. The method of claim 12, wherein the electrode array comprises:
a 1,024 channel array,
wherein the 1,024 channel array is disposed on a thin and flexible substrate,
wherein each channel corresponds to an electrode, and
wherein each electrode has a diameter less than about 500 microns and is spaced from an adjacent electrode of the electrode array by less than about 1 mm.

25. A method for cortical mapping of a brain of a patient comprising:
implanting a pair of electrode arrays proximate to the brain of the patient at first and second positions, respectively, wherein each of the pair of electrode arrays comprises a flexible substrate and a plurality of electrodes arranged on the flexible substrate, wherein the pair of electrode arrays are configured to function together; and
monitoring electrophysiological activity of the brain by:
causing the patient to perform or imagine performing an action over a period of time,
recording, via the pair of electrode arrays, neural activity exhibited by the patient in response to the action performed or imagined by the patient,
decoding the neural activity to determine a correspondence between the neural activity and the action, and
determining a level of confidence, wherein the level of confidence is based on the correspondence, and
recording in a map generated via the cortical mapping, in association with the first and second positions, both the level of confidence and the correspondence between the neural activity and the action.

26. A method for cortical mapping of a brain of a patient comprising:
implanting a pair of electrode arrays proximate to the brain of the patient at first and second positions, respectively, wherein each of the pair of electrode arrays comprises a flexible substrate and a plurality of electrodes arranged on the flexible substrate, wherein the pair of electrode arrays are configured to function together;
stimulating the pair of electrode arrays electrically;
generating an electrical response from the pair of electrode arrays based on the stimulating;
generating a physiologic or behavioral response based on the electrical response;
recording, via the pair of electrode arrays, neural activity exhibited by the patient in response to the physiologic or behavioral response;
decoding the neural activity to determine a correspondence between the neural activity and the physiologic or behavioral response, and
determining a level of confidence, wherein the level of confidence is based on the correspondence, and
recording in a map generated via the cortical mapping, in association with the first and second positions, both the level of confidence and the correspondence between the neural activity and the physiologic or behavioral response.
